(19) United States
(12) Patent Application Publication
(10) Pub. No.: US 2011/0118343 A1
(43) Pub. Date: May 19, 2011

(54) Antitumoral Macrolides

(75) Inventors: José Fernando Reyes Benítez, Madrid (ES); Gloria Crespo Sueiro, Madrid (ES); Rogelio Fernández Rodríguez, Madrid (ES); Andrés Francesch Solloso, Madrid (ES); María del Carmen Cuevas Marchante, Madrid (ES)

(73) Assignee: Pharma Mar, S. A., Madrid (ES)

(21) Appl. No.: 13/001,898

(22) PCT Filed: Jul. 2, 2009
(86) PCT No.: PCT/EP2009/058352

§ 371 Date: Dec. 29, 2010

(30) Foreign Application Priority Data
Jul. 3, 2008 (EP) 08380195.1

(51) Int. Cl. A61K 31/366; C07D 498/18; A61P 35/00

(52) U.S. Cl. 514/455; 540/456

(57) ABSTRACT

Antitumoral compounds of general formula I:

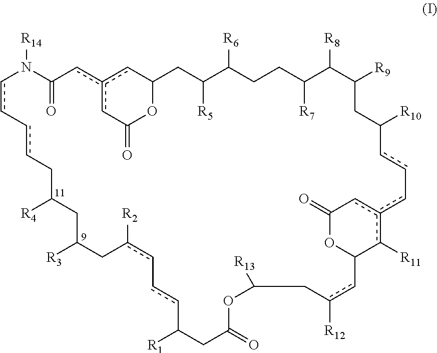

wherein R₁-R₁₄ and the [symbol] lines take permitted meanings for use in the treatment of cancer.

### FIELD OF THE INVENTION

The present invention relates to new antitumoral compounds, pharmaceutical compositions containing them and their use as antitumoral agents.

### BACKGROUND OF THE INVENTION

Several macrolides have been disclosed to have antitumor, antiviral and/or antifungal properties. Specifically, Kitagawa et al. reported the isolation of a symmetrical dimeric macrolide, Swinholide A, from an Okinawan sample of Theonella swinhoei which showed cytotoxic activity (Tetrahedron Lett., 1989, 30, 2963).

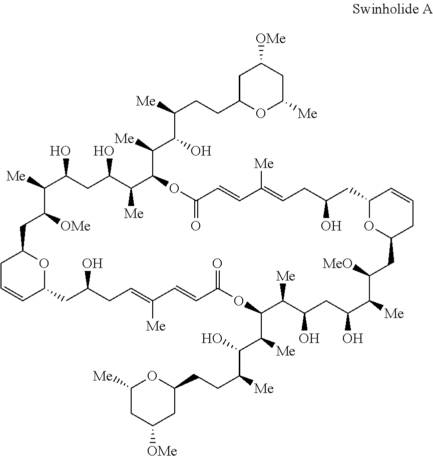

In 1994, Kitagawa et al. disclosed the isolation of new swinholides and a structure-activity study of Swinholide A and its isomers. In this study, Swinholides A, B and C showed to have a potent cytotoxicity against L1210 and KB cell lines with IC₅₀ values of 0.03, 0.30 and 0.14 μg/mL (for L2110) and 0.04, 0.04 and 0.05 μg/mL (for KB), respectively (Chem. Pharm. Bull., 1994, 42(1), 19-26). In addition, it was found that isoswinholide A showed lower cytotoxicity than the other previously mentioned macrolides (IC₅₀ of 1.35 μg/mL for L2110 and 1.1 μg/mL for KB).

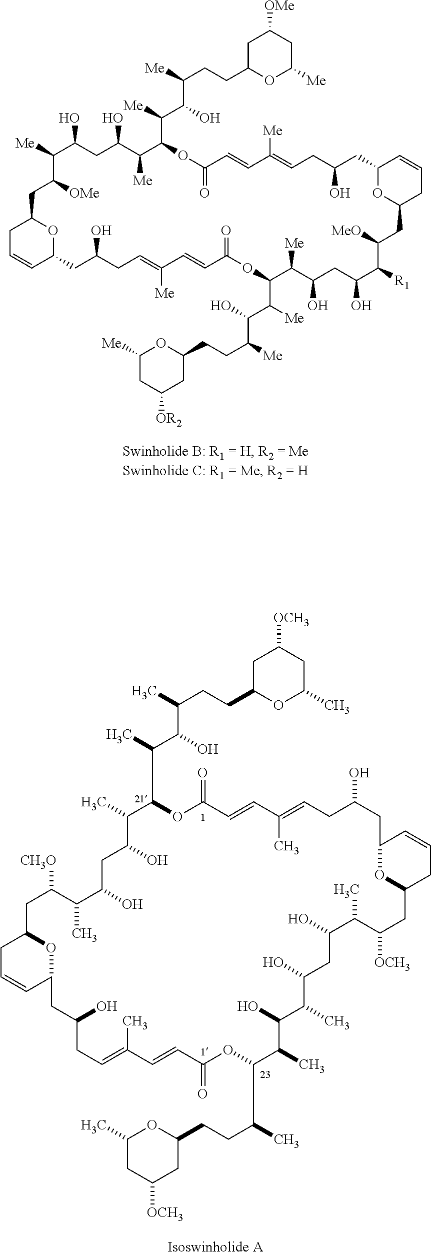

Kitagawa et al. also examined the cytotoxicity of several dimers derived from Swinholide A:

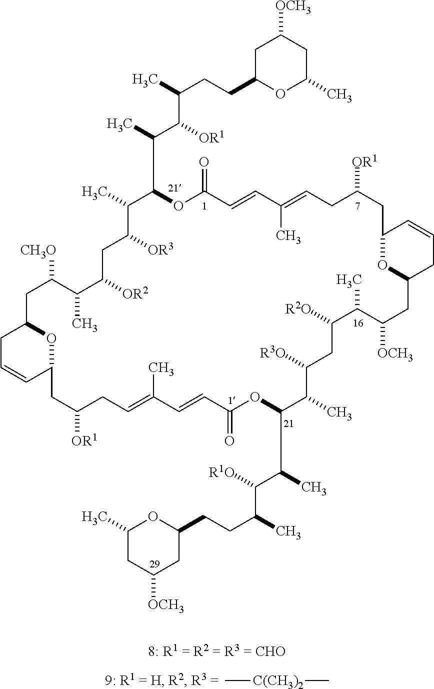

observing that both dimers (8 and 9) show scarce growth inhibitory power in KB cells (51.1% inhibition at 50 μg/mL and 19.3% inhibition at 10 μg/mL, respectively).

Other dimeric macrolides obtained from Swinholide A were the following:

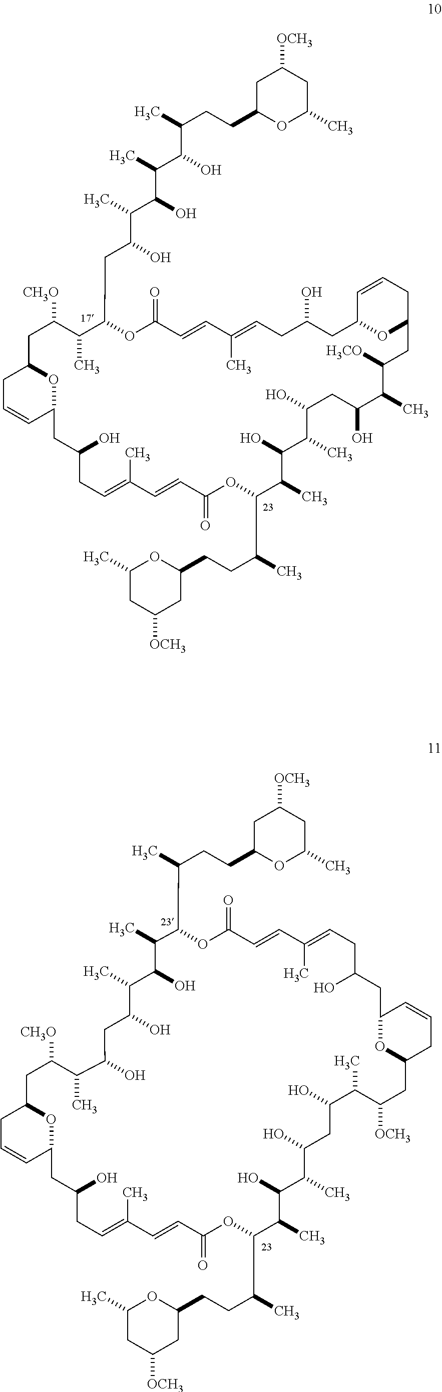

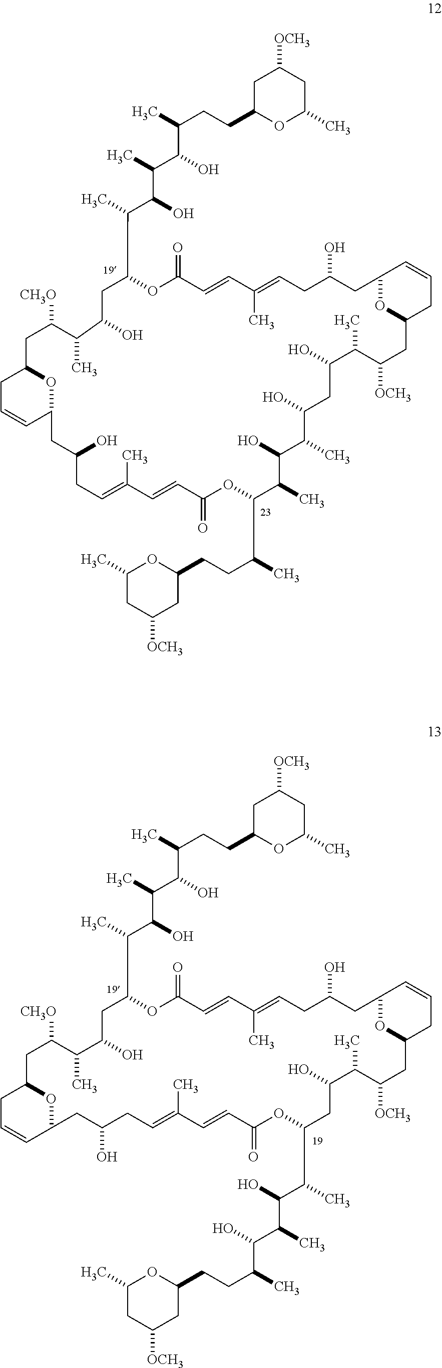

The cytotoxicity of these compounds (10-13) against L1210 and KB cells was lower than the cytotoxicity shown by Swinholide A.

Simultaneously, Kitagawa et al. studied the antitumoral effect of

Swinholide A and its isomers against P388 leukemia cell line in CDF1 mice. Unexpectedly, Swinholide A, isoswinholide A and the isomer (11) were toxic and did not show promising antitumor activity.

In addition, patent application WO 88/00195 describes several macrolides (Misakinolide A (14) and derivatives (15)), which were extracted from a marine sponge of the genus Theonella:

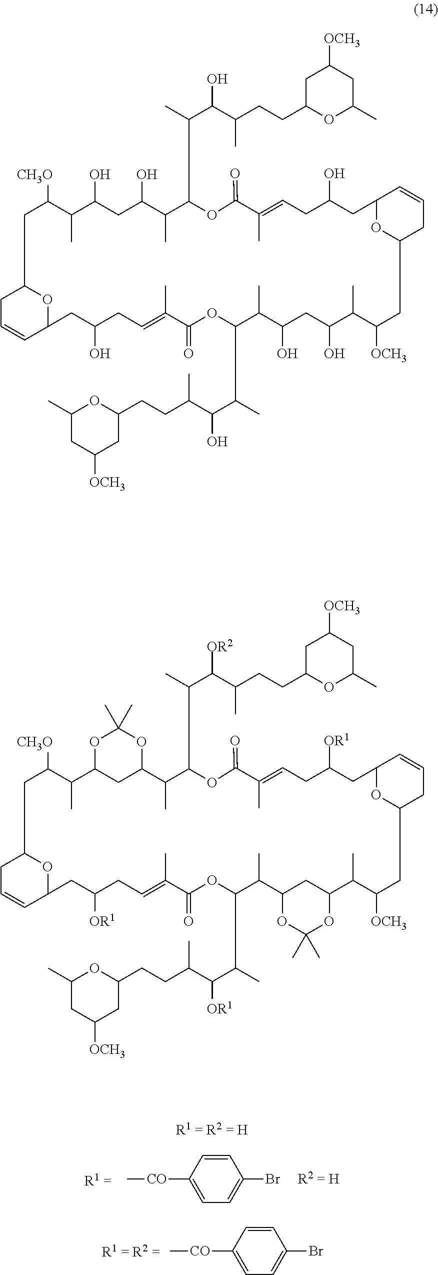

In said patent application, in vitro antitumor activity of Misakinolide A (14) against P388, HCT-8, A549 and MDA-MB-231 cancer cells is described. Likewise, it has also been described that in addition to having a potent cytotoxicity (IC₅₀ 0.035 μg/mL (L1210)), Misakinolide A also has antitumor activity (T/C 140% at a dose of 0.1 mg/kg (mouse) against P388 leukemia) (Chem. Pharm. Bull., 1994, 42(1), 19-26).

Finally, patent application WO 2007/068776 discloses macrolides of general formula (I)

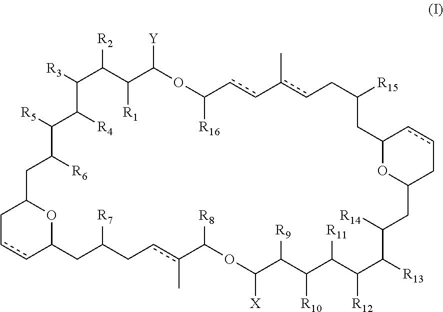

having antitumor activity. Specifically, it is disclosed that compound a, isolated from a sample of Theonella swinhoei, shows a potent cytotoxic activity against HT29, MDA-MB-231, and A549 cell lines with GI₅₀ values of 3.38E-7 M, 8.08E-7 M, and 2.28E-7 M, respectively.

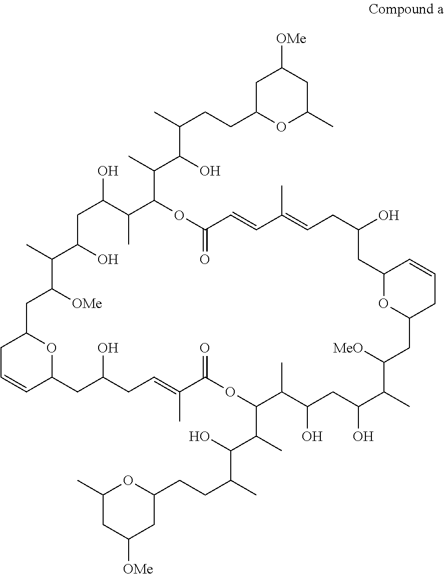

Since cancer is a leading cause of death in animals and humans, several efforts have been and are still being undertaken in order to obtain an antitumor agent active and safe to be administered to patients suffering from a cancer. The problem to be solved by the present invention is to provide compounds that are useful in the treatment of cancer.

### SUMMARY OF THE INVENTION

In one aspect, the present invention is directed to compounds of general formula I or pharmaceutically acceptable salts, tautomers, prodrugs or stereoisomers thereof

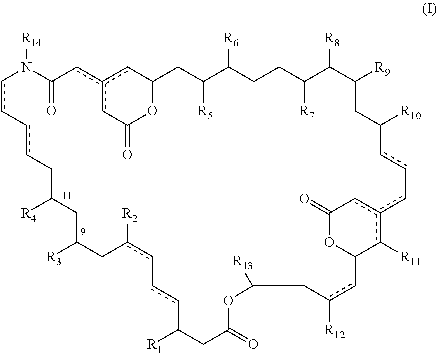

wherein
each R₁, R₃, R₅, R₇, R₉, and R₁₀ is independently selected from hydrogen, halogen, ORₐ, OCORₐ, OCOORₐ, OCONRₐR_{b}, OSO₂Rₐ, OSO₃Rₐ, and ═O, with the proviso that when a ═O group exists the hydrogen of the C atom to which the ═O is attached is absent;
each R₂, R₄, R₆, R₈, R₁₁, R₁₂, and R₁₃ is independently selected from hydrogen, CORₐ, COORₐ, CONRₐR_{b}, substituted or unsubstituted C₁-C₁₂ alkyl, substituted or unsubstituted C₂-C₁₂ alkenyl, and substituted or unsubstituted C₂-C₁₂ alkynyl; or R₃ and R₄ together with the corresponding C atoms to which they are attached and their adjacent C atom form a 5 or 6 membered lactone or lactam ring;
R₁₄ is independently selected from hydrogen, CORₐ, COORₐ, CONRₐR_{b}, ORₐ, OCORₐ, substituted or unsubstituted C₁-C₁₂ alkyl, substituted or unsubstituted C₂-C₁₂ alkenyl, and substituted or unsubstituted C₂-C₁₂ alkynyl;
each Rₐ and R_{b} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
each [symbol] line represents a single or double bond, with the proviso that when one carbon atom bears more than one [symbol] line one of these lines can be a double bond but the others are single bonds.

In another aspect, the present invention is directed to compounds of formula I, or pharmaceutically acceptable salts, tautomers, prodrugs or stereoisomers thereof, for use as a medicament, in particular as a medicament for treating cancer.

In a further aspect, the present invention is also directed to the use of compounds of formula I, or pharmaceutically acceptable salts, tautomers, prodrugs or stereoisomers thereof, in the treatment of cancer, or in the preparation of a medicament, preferably for the treatment of cancer. Other aspects of the invention are methods of treatment, and compounds for use in these methods. Therefore, the present invention further provides a method of treating a patient, notably a human, affected by cancer which comprises administering to said affected individual in need thereof a therapeutically effective amount of a compound as defined above.

In a yet further aspect, the present invention is also directed to compounds of formula I, or pharmaceutically acceptable salts, tautomers, prodrugs or stereoisomers thereof, for use as anticancer agents.

In another aspect, the present invention is directed to pharmaceutical compositions comprising a compound of formula I, or pharmaceutically acceptable salts, tautomers, prodrugs or stereoisomers thereof, together with a pharmaceutically acceptable carrier or diluent.

The present invention also relates to the isolation of compounds of formula I from a porifera of the family Polymastiidae, genus Polymastia, species Polymastia littoralis, and the formation of derivatives from the isolated compounds.

### DETAILED DESCRIPTION OF THE INVENTION

The present invention relates to compounds of general formula I as defined above.

In these compounds the groups can be selected in accordance with the following guidance:

Alkyl groups may be branched or unbranched, and preferably have from 1 to about 12 carbon atoms. One more preferred class of alkyl groups has from 1 to about 6 carbon atoms. Even more preferred are alkyl groups having 1, 2, 3 or 4 carbon atoms. Methyl, ethyl, n-propyl, iso-propyl and butyl, including n-butyl, tert-butyl, sec-butyl and iso-butyl are particularly preferred alkyl groups in the compounds of the present invention. As used herein, the term alkyl, unless otherwise stated, refers to both cyclic and noncyclic groups, although cyclic groups will comprise at least three carbon ring members.

Preferred alkenyl and alkynyl groups in the compounds of the present invention may be branched or unbranched, have one or more unsaturated linkages and from 2 to about 12 carbon atoms. One more preferred class of alkenyl and alkynyl groups has from 2 to about 6 carbon atoms. Even more preferred are alkenyl and alkynyl groups having 2, 3 or 4 carbon atoms. The terms alkenyl and alkynyl as used herein refer to both cyclic and noncyclic groups, although cyclic groups will comprise at least three carbon ring members.

Suitable aryl groups in the compounds of the present invention include single and multiple ring compounds, including multiple ring compounds that contain separate and/or fused aryl groups. Typical aryl groups contain from 1 to 3 separated or fused rings and from 6 to about 18 carbon ring atoms. Preferably aryl groups contain from 6 to about 10 carbon ring atoms. Specially preferred aryl groups include substituted or unsubstituted phenyl, substituted or unsubstituted naphthyl, substituted or unsubstituted biphenyl, substituted or unsubstituted phenanthryl and substituted or unsubstituted anthryl.

Suitable heterocyclic groups include heteroaromatic and heteroalicyclic groups containing from 1 to 3 separated and/or fused rings and from 5 to about 18 ring atoms. Preferably heteroaromatic and heteroalicyclic groups contain from 5 to about 10 ring atoms. Suitable heteroaromatic groups in the compounds of the present invention contain one, two or three heteroatoms selected from N, O or S atoms and include, e.g., coumarinyl including 8-coumarinyl, quinolyl including 8-quinolyl, isoquinolyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothienyl, benzothiazolyl, benzoxazolyl, quinazolinyl, quinoxalinyl, naphthyridinyl, and furopyridinyl. Suitable heteroalicyclic groups in the compounds of the present invention contain one, two or three heteroatoms selected from N, O or S atoms and include, e.g., pyrrolidinyl, tetrahydrofuryl, dihydrofuryl, tetrahydrothienyl, tetrahydrothiopyranyl, piperidyl, morpholinyl, thiomorpholinyl, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, pyrazolidinyl, imidazolinyl, imidazolidinyl, 3-azabicyclo[3.1.0]hexyl, 3-azabicyclo[4.1.01heptyl, 3H-indolyl, and quinolizinyl.

The groups above mentioned may be substituted at one or more available positions by one or more suitable groups such as OR′, ═O, SR′, SOR′, SO₂R′, NO₂, NHR′, N(R′)₂, ═N—R′, N(R′)COR′, N(COR′)₂, N(R′)SO₂R′, N(R′)C(═NR′)N(R′)R′, CN, halogen, COR′, COOR′, OCOR′, OCOOR′, OCONHR′, OCON(R′)₂, CONHR′, CON(R′)₂, CON(R′)OR′, CON(R′)SO₂R′, PO(OR′)₂, PO(OR′)R′, PO(OR′)(N(R′)R′), substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, COOH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list.

Suitable halogen groups or substituents in the compounds of the present invention include F, Cl, Br and I.

The term “pharmaceutically acceptable salts refers to any salt which, upon administration to the patient is capable of providing (directly or indirectly) a compound as described herein. It will be appreciated that non-pharmaceutically acceptable salts also fall within the scope of the invention since those may be useful in the preparation of pharmaceutically acceptable salts. The preparation of salts can be carried out by methods known in the art.

For instance, pharmaceutically acceptable salts of compounds provided herein are synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of both. Generally, nonaqueous media like ether, ethyl acetate, ethanol, 2-propanol or acetonitrile are preferred. Examples of the acid addition salts include mineral acid addition salts such as, for example, hydrochloride, hydrobromide, hydroiodide, sulfate, nitrate, phosphate, and organic acid addition salts such as, for example, acetate, trifluoroacetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

The compounds of the invention may be in crystalline form either as free compounds or as solvates (e.g. hydrates, alcoholates, particularly methanolates) and it is intended that both forms are within the scope of the present invention. Methods of solvation are generally known within the art. The compounds of the invention may present different polymorphic forms, it is intended that the invention encompasses also such forms.

Any compound that is a prodrug of a compound of formula I is within the scope of the invention. The term “prodrug” is used in its broadest sense and encompasses those derivatives that are converted in vivo to the compounds of the invention. Examples of prodrugs include, but are not limited to, derivatives and metabolites of the compounds of formula I that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger “Medicinal Chemistry and Drug Discovery 6^{th} ed. (Donald J. Abraham ed., 2001, Wiley) and “Design and Applications of Prodrugs” (H. Bundgaard ed., 1985, Harwood Academic Publishers).

Any compound referred to herein is intended to represent such specific compound as well as certain variations or forms. In particular, compounds referred to herein may have asymmetric centres and therefore exist in different enantiomeric forms. All optical isomers and stereoisomers of the compounds referred to herein, and mixtures thereof, are considered within the scope of the present invention. Thus any given compound referred to herein is intended to represent any one of a racemate, one or more enantiomeric forms, one or more diastereomeric forms, one or more atropisomeric forms, and mixtures thereof. Particularly, the compounds of the present invention represented by the above described formula I may include enantiomers depending on their asymmetry or diastereoisomers. Stereoisomerism about the double bond is also possible, therefore in some cases the molecule could exist as (E)-isomer or (Z)-isomer. If the molecule contains several double bonds, each double bond will have its own stereoisomerism, that could be the same as, or different to, the stereoisomerism of the other double bonds of the molecule. The single isomers and mixtures of isomers fall within the scope of the present invention.

Furthermore, any compound referred to herein may exist as tautomers. Specifically, the term tautomer refers to one of two or more structural isomers of a compound that exist in equilibrium and are readily converted from one isomeric form to another. Common tautomeric pairs are amine-imine, amide-imidic acid, keto-enol, lactam-lactim, etc. Additionally, any compound referred to herein is intended to represent hydrates, solvates, and polymorphs, and mixtures thereof when such forms exist in the medium. In addition, compounds referred to herein may exist in isotopically-labelled forms. All geometric isomers, tautomers, atropisomers, hydrates, solvates, polymorphs, and isotopically labelled forms of the compounds referred to herein, and mixtures thereof, are considered within the scope of the present invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

In compounds of general formula I, each R₁, R₅, R₇, R₉, and R₁₀ is preferably and independently selected from ORₐ, OCORₐ, and OCOORₐ, wherein Rₐ is selected from hydrogen and substituted or unsubstituted C₁-C₁₂ alkyl. Particularly preferred Rₐ is hydrogen and substituted or unsubstituted C₁-C₆ alkyl; and even more preferred is hydrogen, methyl, ethyl, n-propyl, iso-propyl and butyl, including n-butyl, tert-butyl, sec-butyl and iso-butyl. More preferably, R₁, R₅, R₇, R₉, and R₁₀ are ORₐ, wherein Rₐ is independently selected from hydrogen and substituted or unsubstituted C₁-C₆ alkyl; and even more preferably Rₐ is independently selected from hydrogen, methyl, ethyl, n-propyl, iso-propyl and butyl, including n-butyl, tert-butyl, sec-butyl and iso-butyl. Methoxy is the most preferred R₁, R₅, and R₇ groups, and hydroxy is the most preferred R₉ and R₁₀ groups.

Particularly preferred R₂, R₆, R₈, R₁₁, and R₁₂ are each independently selected from hydrogen and substituted or unsubstituted C₁-C₁₂ alkyl. More preferably R₂, R₆, R₈, R₁₁, and R₁₂ are each independently selected from hydrogen and substituted or unsubstituted C₁-C₆ alkyl. Even more preferably R₂, R₆, R₈, R₁₁, and R₁₂ are each independently selected from methyl, ethyl, n-propyl, iso-propyl and butyl, including n-butyl, tert-butyl, sec-butyl and iso-butyl; being methyl the most preferred.

R₁₃ is preferably selected from hydrogen and substituted or unsubstituted C₁-C₁₂ alkyl. More preferably Rn is substituted or unsubstituted C₁-C₆ alkyl. It is particularly preferred that the alkyl group is substituted by one or more suitable substituents, being the substituents preferably selected from OR′, SR′, NHR′, N(R′)₂, NHCOR′, N(COR′)₂, halogen, OCOR′, OCOOR′, OCONHR′, and OCON(R′)₂, wherein each of the R′ groups is independently selected from hydrogen, substituted or unsubstituted C₁-C₆ alkyl, substituted or unsubstituted C₂-C₆ alkenyl, and substituted or unsubstituted C₂-C₆ alkynyl; and even more preferred the substituent is OR′ wherein R′ is unsubstituted C₁-C₆ alkyl. Most preferred R₁₃ is a substituted methyl; being methoxymethyl the most preferred group.

Particularly preferred R₁₄ is selected from hydrogen and substituted or unsubstituted C₁-C₁₂ alkyl. More preferably R₁₄ is selected from hydrogen and substituted or unsubstituted C₁-C₆ alkyl. Even more preferably R₁₄ is independently selected from hydrogen, methyl, ethyl, n-propyl, iso-propyl and butyl, including n-butyl, tert-butyl, sec-butyl and iso-butyl; being hydrogen the most preferred.

Particularly preferred R₃ is selected from ORₐ, OCORₐ, and OCOORₐ, wherein Rₐ is selected from hydrogen and substituted or unsubstituted C₁-C₁₂ alkyl. Particularly preferred Rₐ is hydrogen and substituted or unsubstituted C₁-C₆ alkyl; and even more preferred is hydrogen, methyl, ethyl, n-propyl, iso-propyl and butyl, including n-butyl, tert-butyl, sec-butyl and iso-butyl. More preferably, R₃ is ORₐ, wherein Rₐ is independently selected from hydrogen and substituted or unsubstituted C₁-C₆ alkyl; and even more preferably Rₐ is independently selected from hydrogen, methyl, ethyl, n-propyl, iso-propyl and butyl, including n-butyl, tert-butyl, sec-butyl and iso-butyl. Hydroxy is the most preferred R₃ group.

Particularly preferred R₄ is selected from hydrogen and substituted or unsubstituted C₁-C₁₂ alkyl. More preferably R₄ is a substituted or unsubstituted C₁-C₆ alkyl. It is particularly preferred that the alkyl group is substituted by one or more suitable substituents, being the substituents preferably selected from SO₂R′, COR′, COOR′, CONHR′, CON(R′)₂, CON(R′)OR′, CON(R′)SO₂R′, PO(OR′)₂, PO(OR′)R′, PO(OR′)(N(R′)R′), and substituted or unsubstituted heterocyclic group, wherein each of the R′ groups is independently selected from hydrogen, substituted or unsubstituted C₁-C₆ alkyl, substituted or unsubstituted C₂-C₆ alkenyl, and substituted or unsubstituted C₂-C₆ alkynyl; and even more preferred the substituent is COOR′ wherein R′ is unsubstituted C₁-C₆ alkyl. Most preferred R₄ is a substituted methyl; being methoxycarbonylmethyl the most preferred group.

In another preferred class of compounds of the invention, R₃ and R₄ together with the corresponding C atoms to which they are attached and their adjacent C atom form a 5 or 6 membered lactone ring. A 6 membered lactone ring of formula

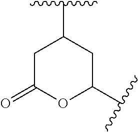

is more preferred; and even more preferred is a 6 membered lactone ring of formula

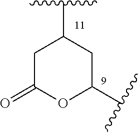

wherein the labeled C atoms correspond with their homonyms in formula I.

Particularly preferred is that each [symbol] line is a double bond, with the proviso that when one carbon atom bears more than one [symbol] line one of these lines is a double bond and the others are single bonds. More preferred is that each [symbol] line is a double bond, with the proviso that when one carbon atom bears more than one [symbol] line one of these lines is a double bond and the others are single bonds, and at least one lactone ring has a double bond conjugated with its carbonyl group.

More particularly, the invention provides compounds of general formula II or pharmaceutically acceptable salts, tautomers, prodrugs or stereoisomers thereof

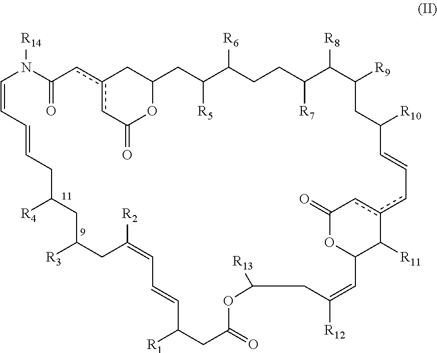

wherein R₁-R₁₄ groups and the [symbol] lines have the same meaning given above.

In compounds of general formula II, each R₁, R₅, R₇, R₉, and R₁₀ is preferably and independently selected from ORₐ, OCORₐ, and OCOORₐ, wherein Rₐ is selected from hydrogen and substituted or unsubstituted C₁-C₁₂ alkyl. Particularly preferred Rₐ is hydrogen and substituted or unsubstituted C₁-C₆ alkyl; and even more preferred is hydrogen, methyl, ethyl, n-propyl, iso-propyl and butyl, including n-butyl, tert-butyl, sec-butyl and iso-butyl. More preferably, R₁, R₅, R₇, R₉, and R₁₀ are ORₐ, wherein Rₐ is independently selected from hydrogen and substituted or unsubstituted C₁-C₆ alkyl; and even more preferably Rₐ is independently selected from hydrogen, methyl, ethyl, n-propyl, iso-propyl and butyl, including n-butyl, tert-butyl, sec-butyl and iso-butyl. Methoxy is the most preferred R₁, R₅, and R₇ groups, and hydroxy is the most preferred R₉ and R₁₀ groups.

Particularly preferred R₂, R₆, R₈, R₁₁, and R₁₂ are each independently selected from hydrogen and substituted or unsubstituted C₁-C₁₂ alkyl. More preferably R₂, R₆, R₈, R₁₁, and R₁₂ are each independently selected from hydrogen and substituted or unsubstituted C₁-C₆ alkyl. Even more preferably R₂, R₆, R₈, R₁₁, and R₁₂ are each independently selected from methyl, ethyl, n-propyl, iso-propyl and butyl, including n-butyl, tert-butyl, sec-butyl and iso-butyl; being methyl the most preferred.

R₁₃ is preferably selected from hydrogen and substituted or unsubstituted C₁-C₁₂ alkyl. More preferably R₁₃ is substituted or unsubstituted C₁-C₆ alkyl. It is particularly preferred that the alkyl group is substituted by one or more suitable substituents, being the substituents preferably selected from OR′, SR′, NHR′, N(R′)₂, NHCOR′, N(COR′)₂, halogen, OCOR′, OCOOR′, OCONHR′, and OCON(R′)₂, wherein each of the R′ groups is independently selected from hydrogen, substituted or unsubstituted C₁-C₆ alkyl, substituted or unsubstituted C₂-C₆ alkenyl, and substituted or unsubstituted C₂-C₆ alkynyl; and even more preferred the substituent is OR′ wherein R′ is unsubstituted C₁-C₆ alkyl. Most preferred R₁₃ is a substituted methyl; being methoxymethyl the most preferred group.

Particularly preferred R₁₄ is selected from hydrogen and substituted or unsubstituted C₁-C₁₂ alkyl. More preferably R₁₄ is selected from hydrogen and substituted or unsubstituted C₁-C₆ alkyl. Even more preferably R₁₄ is independently selected from hydrogen, methyl, ethyl, n-propyl, iso-propyl and butyl, including n-butyl, tert-butyl, sec-butyl and iso-butyl; being hydrogen the most preferred.

Particularly preferred R₃ is selected from ORₐ, OCORₐ, and OCOORₐ, wherein Rₐ is selected from hydrogen and substituted or unsubstituted C₁-C₁₂ alkyl. Particularly preferred Rₐ is hydrogen and substituted or unsubstituted C₁-C₆ alkyl; and even more preferred is hydrogen, methyl, ethyl, n-propyl, iso-propyl and butyl, including n-butyl, tert-butyl, sec-butyl and iso-butyl. More preferably, R₃ is ORₐ, wherein Rₐ is independently selected from hydrogen and substituted or unsubstituted C₁-C₆ alkyl; and even more preferably Rₐ is independently selected from hydrogen, methyl, ethyl, n-propyl, iso-propyl and butyl, including n-butyl, tert-butyl, sec-butyl and iso-butyl. Hydroxy is the most preferred R₃ group.

Particularly preferred R₄ is selected from hydrogen and substituted or unsubstituted C₁-C₁₂ alkyl. More preferably R₄ is a substituted or unsubstituted C₁-C₆ alkyl. It is particularly preferred that the alkyl group is substituted by one or more suitable substituents, being the substituents preferably selected from SO₂R′, COR′, COOR′, CONHR′, CON(R′)₂, CON(R′)OR′, CON(R′)SO₂R′, PO(OR′)₂, PO(OR′)R′, PO(OR′)(N(R′)R′), and substituted or unsubstituted heterocyclic group, wherein each of the R′ groups is independently selected from hydrogen, substituted or unsubstituted C₁-C₆ alkyl, substituted or unsubstituted C₂-C₆ alkenyl, and substituted or unsubstituted C₂-C₆ alkynyl; and even more preferred the substituent is COOR′ wherein R′ is unsubstituted C₁-C₆ alkyl. Most preferred R₄ is a substituted methyl; being methoxycarbonylmethyl the most preferred group.

In another preferred class of compounds of the invention, R₃ and R₄ together with the corresponding C atoms to which they are attached and their adjacent C atom form a 5 or 6 membered lactone ring. A 6 membered lactone ring of formula

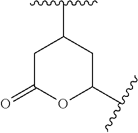

is more preferred; and even more preferred is a 6 membered lactone ring of formula

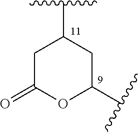

wherein the labeled C atoms correspond with their homonyms in formula II.

Particularly preferred is that each [symbol] line is a double bond, with the proviso that when one carbon atom bears more than one [symbol] line one of these lines is a double bond and the other is a single bond. More preferred is that each [symbol] line is a double bond, with the proviso that when one carbon atom bears more than one [symbol] line one of these lines is a double bond and the other is a single bond, and at least one lactone ring has a double bond conjugated with its carbonyl group.

In additional preferred embodiments, the preferences described above for the different substituents are combined. The present invention is also directed to such combinations of preferred substitutions in the formula I or II above.

In the present description and definitions, when there are several substituents Rₐ or R_{b} present in the compounds of the invention, and unless it is stated explicitly so, it should be understood that they can be each independently different within the given definition, i.e. Rₐ does not represent necessarily the same group simultaneously in a given compound of the invention.

Particularly preferred compounds of the invention are those having the following formulae:

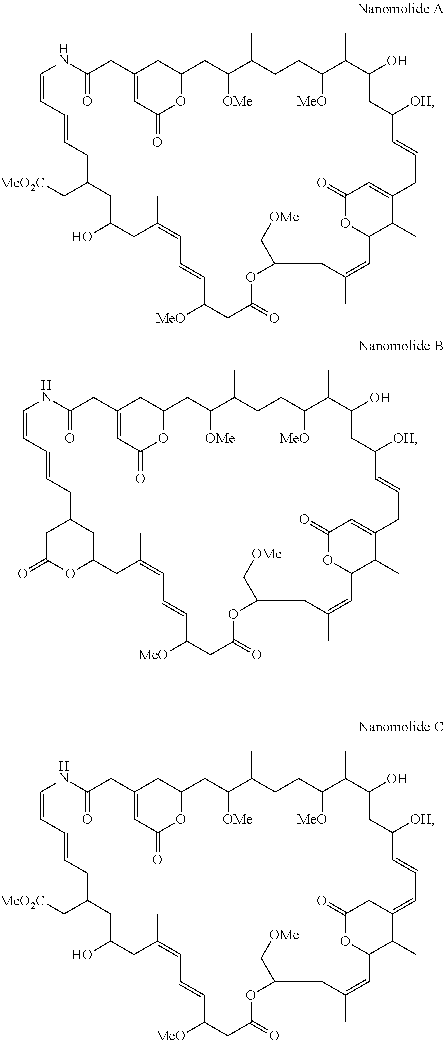

or pharmaceutically acceptable salts, tautomers, prodrugs or stereoisomers thereof.

Nanomolides A-C were isolated from a porifera, of the order Hadromerida, family Polymastiidae, genus Polymastia, species Polymastia littoralis. Polymastia littoralis was originally described in 1915 by Stephens (Transactions of the Royal Society of Edinburgh 50(2): 423-467, pls XXXVIII-XL). A sample of Polymastia littoralis was deposited in the Institute of Marine Sciences and Limnology of

Universidad Nacional Autónoma of Mexico, with the reference code SHIM-565. This sponge was collected by hand using SCUBA diving in Shimoni Channel, Mombasa, Kenya (04° 40.576′ S/39° 26.182′ E) at depths ranging between 27 and 30 m.

The description of this sponge is the following: Encrusting and cushion-shaped sponge, of approximately 1 cm thick in average, 5×1 cm in diameter, with papilla up to 0.6 mm long, and approximately 1-3 mm in diameter. When alive, its color is brown, and when preserved in alcohol its color is beige. Its cortex consists of small styles that form a palisade of approximately 100 to 150 μm thick, which barely protrude through the surface. The dense dermal layer of small spicules is about 0.3 mm in thickness. Choanosomal skeleton consists of tracts of 100-250 mm wide, which arise from the sponge base to the cortex. A few vertical choanosomal tracts penetrate the cortex and project slightly beyond the surface of the sponge. Ectosomal styles are straight of 503 μm long, with very slim heads from 87 to 150 μm in average. Choanosomal styles are smooth, straight uniform in diameter, from 500 to 850 μm long in average, with slim heads.

Additionally, compounds of the invention can be obtained by synthesis following usual procedures in synthetic organic chemistry and already known by a person skilled in the art. For example, compounds of this invention can be obtained adapting the procedures described in the literature: M. B. Smith, J. March in March's Advanced Organic Chemistry, 6^{th} ed., John Wiley and Sons, Inc., New York, 2007; Comprehensive Organic Synthesis, B. M. Trost, editor-in-chief, Pergamon Press, Oxford 1991; Carey, Organic Chemistry, 6^{th} ed., McGraw-Hill, New York, 2006; Larock, Comprehensive Organic Transformations, 2^{nd} ed. Wiley-VCH, New York, 1999.

Likewise, natural, synthetic or already modified compounds of the invention can be further modified by a variety of chemical reactions to obtain additional compounds of the invention. Thus, hydroxyl groups can be acylated by standard coupling or acylation procedures, for instance by using acetic acid, acetyl chloride or acetic anhydride in pyridine or the like. Formate groups can be obtained by heating hydroxyl precursors in formic acid. Carbamates can be obtained by heating hydroxyl precursors with isocyanates. Hydroxyl groups can be converted into halogen groups through intermediate sulfonates for iodide, bromide or chloride, or directly using a (diethylamino)sulfur trifluoride for fluorides; or they can be reduced to hydrogen by reduction of intermediate sulfonates. Hydroxyl groups can also be converted into alkoxy groups by alkylation using an alkyl bromide, iodide or sulfonate, or into amino lower alkoxy groups by using, for instance, a protected 2-bromoethylamine. Amido groups can be alkylated or acylated by standard alkylation or acylation procedures, for instance by using, respectively, KH and methyl iodide or acetyl chloride in pyridine or the like. Ester groups can be hydrolized to carboxylic acids or reduced to aldehyde or to alcohol. Carboxylic acids can be coupled with amines to provide amides by standard coupling or acylation procedures.

When necessary, appropriate protecting groups can be used on the substituents to ensure that reactive groups are not affected. These protecting groups are well known for the skilled person in the art. A general review of protecting groups in organic chemistry is provided by Wuts, P. G. M. and Greene T. W. in Protecting groups in Organic Synthesis, 4^{th} Ed. Wiley-Interscience, and by Kocienski P. J. in Protecting Groups, 3^{rd} Ed. Georg Thieme Verlag. All these references are incorporated by reference in their entirety.

The synthesis can be designed to employ precursor substituents which can be converted at the appropriate stage to a desired substituent. Saturation or unsaturation in the ring-structure can be introduced or removed as part of the synthesis. Starting materials and reagents can be modified as desired to ensure synthesis of the intended compound.

An important feature of the above described compounds of formula I and II is their bioactivity and in particular their cytotoxic activity.

With this invention we provide novel pharmaceutical compositions of compounds of general formula I and II that possess cytotoxic activities and their use as antitumor agents. Thus the present invention further provides pharmaceutical compositions comprising a compound of this invention, or a pharmaceutically acceptable salt, tautomer, prodrug or stereoisomer thereof with a pharmaceutically acceptable carrier or diluent.

The term “carrier” refers to an adjuvant, excipient or vehicle with which the active ingredient is administered. Suitable pharmaceutical carriers are described in “Remington's Pharmaceutical Sciences” by E. W. Martin, 1995.

Examples of pharmaceutical compositions include any solid (tablets, pills, capsules, granules etc.) or liquid (solutions, suspensions or emulsions) composition for oral, topical or parenteral administration.

Administration of the compounds or compositions of the present invention may be by any suitable method, such as intravenous infusion, oral preparations, and intraperitoneal and intravenous administration. We prefer that infusion times of up to 24 hours are used, more preferably 1-12 hours, with 1-6 hours most preferred. Short infusion times which allow treatment to be carried out without an overnight stay in hospital are especially desirable. However, infusion may be 12 to 24 hours or even longer if required. Infusion may be carried out at suitable intervals of say 1 to 4 weeks. Pharmaceutical compositions containing compounds of the invention may be delivered by liposome or nanosphere encapsulation, in sustained release formulations or by other standard delivery means.

The correct dosage of the compounds will vary according to the particular formulation, the mode of application, and the particular situs, host and tumour being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

As used herein, the terms “treat”, “treating” and “treatment” include the eradication, removal, modification, or control of a tumor or primary, regional, or metastatic cancer cells or tissue and the minimization or delay of the spread of cancer.

The compounds of the invention have anticancer activity against several cancers types which include, but are not limited, lung cancer, colon cancer, and breast cancer.

Thus, in an alternative embodiment of the present invention, the pharmaceutical composition comprising the compounds of formula I or II as defined above is for the treatment of lung cancer, colon cancer, or breast cancer.

### EXAMPLES

### Example 1

### Description of the Marine Organism and Collection Site

Polymastia littoralis was collected by hand using SCUBA diving in Shimoni Channel, Mombasa, Kenya (04° 40.576′ S/39° 26.182′ E) at depths ranging between 27 and 30 m. The animal material was identified by Dr. José Luis Carballo (Universidad Nacional Autónoma of Mexico). A sample of the specimen was deposited in the Institute of Marine Sciences and Limnology of the Universidad Nacional Autónoma of Mexico, with the reference code SHIM-565.

### Example 2

### Isolation of Nanomolide A

The frozen specimen of Example 1 (66 g) was triturated and extracted with a mixture of CH₃OH:CH₂Cl₂ (50:50, 4×300 mL) at 23° C. The combined organic extracts were concentrated to yield a crude of 2.69 g. This material was subjected to VLC on Polygoprep C18 silica gel with a stepped gradient from H₂O to MeOH. Nanomolide A (9.7 mg) was isolated from a fraction eluting with H₂O:MeOH 1:9 (215.4 mg) by semipreparative reversed phase HPLC (Atlantis dC₁₈, 10 μm, 10×150 mm, gradient H₂O:CH3CN from 40 to 61.6% CH₃CN in 18 min, UV detection, flow 4.0 mL/min, retention time 16.5 min).

Nanomolide A: Amorphous colourless solid. (+)-HRMALDITOFMS m/z 1053.6033 M⁺ (calc. for C₅₈H₈₇NO₁₆, 1053.6019), m/z 1076.5938 [M+Na]⁺ (calc. for C₅₈H₈₇NO₆Na, 1076.5917). ¹H (500 MHz) and ¹³C NMR (125 MHz) see Table 1.

TABLE 1
¹H and ¹³C NMR data of Nanomolide A (CD₃OD and Acetone-d₆)

| | CD₃OD | | (CD₃)₂CO | |
|---|---|---|---|---|
| N^{o} | ¹H, m, J (Hz) | ¹³C | ¹H, m, J (Hz) | ¹³C |
| 1 | — | 171.8 | — | 170.5 |
| 2 | 2.58 (dd, 15.0, 7.9) | 42.3 | 2.57 (dd, 15.1, 8.0) | 42.1 |
| | 2.45 (m) | | 2.42 (m) | |
| 3 | 4.05 (m) | 80.0 | 4.05 (ddd, 8.0, 7.3, 6.2) | 79.4 |
| 4 | 5.40 (dd, 15.3, 7.9) | 130.4 | 5.43 (dd, 14.7, 7.3) | 130.4 |
| 5 | 6.51 (dd, 15.3, 11.3) | 131.2 | 6.55 (dd, 14.7, 11.0) | 130.5 |
| 6 | 5.93 (br d, 11.3) | 127.6 | 5.94 (br d, 11.0) | 127.3 |
| 7 | — | 138.7 | — | 138.4 |
| 8 | 2.37 (m) | 42.3 | 2.35 (m) | 42.1 |
| | 2.30 (m) | | | |
| 9 | 3.84 (m) | 68.7 | 3.88 (m) | 68.0 |
| 9-OH | Not observed | — | 3.60 (d, 5.5) | — |
| 10 | 1.47 (m) | 41.7 | 1.47 (m) | 41.7 |
| | 1.33 (m) | | 1.34 (m) | |
| 11 | 2.23 (m) | 33.2 | 2.24 (m) | 32.8 |
| 12 | 2.23 (m) | 39.5 | 2.18 (m) | 39.0 |
| | 2.14 (m) | | 2.10 (m) | |
| 13 | 5.66 (m) | 133.1 | 5.64 (dt, 14.7, 7.2) | 132.1 |
| 14 | 6.45 (dd, 15.1, 11.5) | 126.6 | 6.42 (dd, 14.7, 11.3) | 126.3 |
| 15 | 5.44 (dd, 11.5, 9.4) | 113.5 | 5.39 (dd, 11.3, 8.9) | 111.4 |
| 16 | 6.49 (d, 9.4) | 120.4 | 6.57 (dd, 10.7, 8.9) | 120.8 |
| 17-NH | Not observed | — | 9.26 (d, 10.7) | — |
| 18 | — | 168.1 | — | 166.3 |
| 19 | 3.35 (m) | 44.3* | 3.38 (m) | 44.0 |
| 20 | — | 157.3 | — | 155.7 |
| 21 | 2.48 (m) | 34.4 | 2.53 (m) | 34.1 |
| | | | 2.44 (m) | |
| 22 | 4.65 (dddd, 9.7, 9.7, | 76.5 | 4.59 (dddd, 9.9, 9.9, | 75.5 |
| | 5.1, 3.1) | | 3.9, 3.4) | |
| 23 | 1.80 (m) | 36.2 | 1.80 (m) | 35.8 |
| | 1.61 (m) | | 1.58 (m) | |
| 24 | 3.49 (m) | 81.1 | 3.46 (m) | 80.1 |
| 25 | 1.86 (m) | 35.6 | 1.86 (m) | 35.2 |
| 26 | 1.29 (m) | 28.7 | 1.27 (m) | 28.8 |
| | 1.21 (m) | | | |
| 27 | 1.60 (m) | 28.5 | 1.61 (m) | 28.3 |
| | 1.52 (m) | | 1.56 (m) | |
| 28 | 3.22 (m) | 84.4 | 3.20 (m) | 84.8 |
| 29 | 1.68 (m) | 41.3 | 1.70 (m) | 40.7 |
| 30 | 4.05 (m) | 68.9 | 4.09 (m) | 68.3 |
| 30-OH | Not observed | — | 3.45 (m) | — |
| 31 | 1.63 (m) | 43.4 | 1.67 (m) | 43.2 |
| | 1.53 (m) | | 1.49 (m) | |
| 32 | 4.30 (ddd, 8.6, 4.0, 4.0) | 70.0 | 4.37 (m) | 69.6 |
| 32-OH | Not observed | — | 3.95 (d, 5.2) | — |
| 33 | 5.71 (m) | 139.3 | 5.75 (m) | 139.6 |
| 34 | 5.71 (m) | 125.4 | 5.74 (m) | 124.2 |
| 35 | 3.08 (m) | 38.2 | 3.08 (m) | 37.8 |
| 36 | — | 169.4 | — | 167.3 |
| 37 | 2.36 (m) | 38.2 | 2.38 (m) | 37.8 |
| 38 | 5.25 (dd, 8.9, 3.3) | 78.7 | 5.23 (dd, 8.7, 3.3) | 77.4 |
| 39 | 5.54 (dd, 8.9, 1.1) | 124.7 | 5.54 (dd, 8.7, 1.2) | 124.9 |
| 40 | — | 139.3 | — | 138.1 |
| 41 | 2.42 (m) | 34.5 | 2.44 (m) | 34.3 |
| 42 | 5.10 (m) | 72.4 | 5.12 (m) | 71.5 |
| 43 | 3.46 (m) | 73.9 | 3.47 (dd, 10.6, 5.2) | 73.6 |
| | | | 3.44 (dd, 10.6, 4.1) | |
| 44 | 3.33 (s) | 59.4 | 3.30 (s) | 59.1 |
| 45 | 3.22 (s) | 56.7 | 3.21 (s) | 56.3 |
| 46 | 1.82 (br s) | 24.7 | 1.82 (br s) | 24.6 |
| 47 | 2.39 (m) | 38.8 | 2.40 (m) | 38.4 |
| | 2.27 (m) | | 2.24 (m) | |
| 48 | — | 175.2 | — | 173.8 |
| 49 | 3.65 (s) | 52.0 | 3.61 (s) | 51.5 |
| 50 | 5.91 (br s) | 118.7 | 5.87 (br s) | 118.8 |
| 51 | — | 167.0 | — | 164.7 |
| 52 | 3.36 (s) | 57.8 | 3.33 (s) | 57.4 |
| 53 | 0.88 (d, 6.7) | 14.4 | 0.86 (d, 6.8) | 14.5 |
| 54 | 3.34 (s) | 57.5 | 3.32 (s) | 57.4 |
| 55 | 0.89 (d, 6.9) | 10.2 | 0.91 (d, 7.0) | 10.5 |
| 56 | 5.76 (br s) | 115.4 | 5.70 (br s) | 115.4 |
| 57 | — | 167.8 | — | 165.2 |
| 58 | 1.10 (d, 7.1) | 11.8 | 1.10 (d, 7.1) | 11.8 |
| 59 | 1.85 (d, 1.1) | 24.0 | 1.84 (d, 1.2) | 23.8 |

*Detected by HSQC.

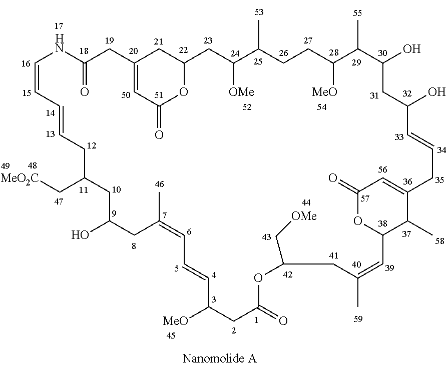

### Example 3

### Isolation of Nanomolide B and Nanomolide C

A second group of samples of the specimen of Example 1 (304.5 g) was triturated and extracted with a mixture of MeOH:CH₂Cl₂ (50:50) at 23° C. The organic extract was evaporated under reduced pressure to yield a crude of 11.85 g. This material was chromatographed (VLC) on Lichroprep RP-18 with a stepped gradient from H₂O to MeOH and CH₂Cl₂. Fraction eluted with H₂O:MeOH 1:9 (621.2 mg) was subjected to preparative reversed phase HPLC (Atlantis Prep dC₁₈, 5 μm, 19×150 mm, gradient H₂O:CH₃CN from 40 to 61.6% of CH₃CN in 18 min, 14.4 mL/min, UV detection) to yield 3 fractions (H1 to H3). Fraction H2 (17-18 min) from this chromatography was subjected to semipreparative HPLC (X-Bridge C18, 5 μm, 10×150 mm, isocratic H₂O:CH₃CN 57:43 in 40 min, 4.0 mL/min, UV detection) to yield Nanomolide A (17.1 mg, retention time 31.6 min), Nanomolide C (2.5 mg, retention time 35.8 min) and a mixture (retention time 30-31 min) that was separated by semipreparative HPLC (X-Terra Phenyl, 5 μm, 10×150 mm, isocratic H₂O:CH₃CN 60:40 in 30 min, 4.0 mL/min, UV detection) to yield a further amount of Nanomolide A (1.6 mg, retention time 25.1 min) and Nanomolide B (0.8 mg, retention time 27.1 min).

Nanomolide B: Amorphous colourless solid. (+)-ESIMS m/z 1060.4 [M+K]⁺, 1044.5 [M+Na]⁺, 990.2 [M−MeOH+H]⁺, 972.3 [M−MeOH−H₂O+H]⁺, 954.3 [M−MeOH−2×H₂O+H]⁺. ¹H (500 MHz) and ¹³C NMR (125 MHz) see Table 2.

Nanomolide C: Amorphous colourless solid. (+)-ESIMS m/z 1076.4 [M+Na]⁺, 1022.5 [M−MeOH+H]⁺, 1004.5 [M−MeOH−H₂O+H]⁺, 986.5 [M−MeOH−2×H₂O+H]⁺. ¹H (500 MHz) and ¹³C NMR (125 MHz) see Table 3.

TABLE 2
¹H and ¹³C NMR data of Nanomolide B (Acetone-d₆).

| N^{o} | ¹H, m, J (Hz) | ¹³C | N^{o} | ¹H, m, J (Hz) | ¹³C |
|---|---|---|---|---|---|
| 1 | — | 170.6 | 30-OH | 3.46 (m) | — |
| 2 | 2.55 (m) | 41.9 | 31 | 1.67 (m) | 43.1 |
| | 2.43 (m) | | | 1.49 (m) | |
| 3 | 4.08 (m) | 79.2 | 32 | 4.37 (m) | 69.6 |
| 4 | 5.51 (dd, 15.2, 7.4) | 131.7 | 32-OH | 3.95 (d, 5.3) | — |
| 5 | 6.57 (dd, 15.2, 11.1) | 129.6 | 33 | 5.74 (m) | 139.6 |
| 6 | 6.00 (br d, 11.1) | 128.3 | 34 | 5.74 (m) | 124.2 |
| 7 | — | 135.9 | 35 | 3.08 (m) | 37.8 |
| 8 | 2.61 (dd, 13.2, 6.5) | 38.5 | 36 | — | 167.2 |
| | 2.53 (m) | | | | |
| 9 | 4.56 (m) | 76.3 | 37 | 2.38 (m) | 37.8 |
| 10 | 1.72 (m) | 32.7 | 38 | 5.22 (dd, 8.7, 3.3) | 77.4 |
| 11 | 2.16 (m) | 29.2 | 39 | 5.53 (br d, 8.7) | 124.9 |
| 12 | 2.20 (m) | 39.6 | 40 | — | 138.1 |
| | 2.14 (m) | | | | |
| 13 | 5.67 (m) | 130.6 | 41 | 2.46 (m) | 34.3 |
| | | | | 2.42 (m) | |
| 14 | 6.49 (dd, 15.0, 11.3) | 126.9 | 42 | 5.14 (m) | 71.5 |
| 15 | 5.40 (dd, 11.3, 9.3) | 111.1 | 43 | 3.46 (m) | 73.7 |
| 16 | 6.60 (dd, 10.7, 9.3) | 121.2 | 44 | 3.30 (s) | 59.1 |
| 17-NH | 9.28 (d, 10.7) | — | 45 | 3.22 (s) | 56.5 |
| 18 | — | 166.3 | 46 | 1.85 (br s) | 24.6 |
| 19 | 3.38 (m) | 44.0 | 47 | 2.47 (m) | 35.8 |
| | | | | 2.30 (dd, 16.1, 9.2) | |
| 20 | — | 155.7 | 48 | — | 171.7 |
| 21 | 2.50 (m) | 34.2 | 49 | 5.87 (br s) | 118.7 |
| | 2.47 (m) | | | | |
| 22 | 4.58 (m) | 75.5 | 50 | — | 164.7 |
| 23 | 1.80 (m) | 35.8 | 51 | 3.33 (s) | 57.4 |
| | 1.58 (m) | | | | |
| 24 | 3.46 (m) | 80.2 | 52 | 0.87 (d, 6.8) | 14.5 |
| 25 | 1.88 (m) | 35.1 | 53 | 3.32 (s) | 57.4 |
| 26 | 1.27 (m) | 28.8 | 54 | 0.91 (d, 7.0) | 10.5 |
| 27 | 1.65 (m) | 28.5 | 55 | 5.69 (br s) | 115.4 |
| | 1.56 (m) | | | | |
| 28 | 3.20 (m) | 84.7 | 56 | — | 165.1 |
| 29 | 1.70 (m) | 40.8 | 57 | 1.10 (d, 7.1) | 11.8 |
| 30 | 4.10 (m) | 68.3 | 58 | 1.84 (d, 1.3) | 23.8 |

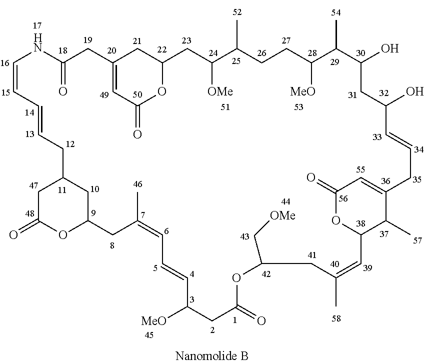

TABLE 3
¹H and ¹³C NMR data of Nanomolide C (Acetone-d₆).

| N^{o} | ¹H, m, J (Hz) | ¹³C | N^{o} | ¹H, m, J (Hz) | ¹³C |
|---|---|---|---|---|---|
| 1 | — | 170.5 | 30-OH | 3.47 (m) | — |
| 2 | 2.57 (dd, 14.8, 7.8) | 42.1 | 31 | 1.69 (m) | 43.1 |
| | 2.44 (m) | | | 1.53 (m) | |
| 3 | 4.06 (m) | 79.4 | 32 | 4.42 (m) | 69.9 |
| 4 | 5.42 (m) | 130.4 | 32-OH | 4.01 (d, 5.2) | — |
| 5 | 6.57 (m) | 130.6 | 33 | 5.82 (dd, 15.1, 5.6) | 139.4 |
| 6 | 5.94 (br d, 11.1) | 127.2 | 34 | 6.46 (dd, 15.1, 11.1) | 124.1 |
| 7 | — | 138.5 | 35 | 6.02 (d, 11.1) | 125.4 |
| 8 | 2.36 (m) | 42.1 | 36 | — | 134.3 |
| 9 | 3.88 (m) | 68.2 | 37 | 2.77 (m) | 40.1 |
| 9-OH | 3.60 (m) | — | 38 | 5.25 (dd, 8.8, 3.2) | 77.8 |
| 10 | 1.51 (m) | 41.8 | 39 | 5.39 (br d, 8.8) | 124.3 |
| | 1.36 (m) | | | | |
| 11 | 2.24 (m) | 32.9 | 40 | — | 139.6 |
| 12 | 2.20 (m) | 39.0 | 41 | 2.53 (m) | 34.2 |
| | 2.12 (m) | | | 2.47 (m) | |
| 13 | 5.64 (dt, 15.1, 7.3) | 132.0 | 42 | 5.14 (m) | 71.4 |
| 14 | 6.43 (m) | 126.4 | 43 | 3.46 (m) | 73.8 |
| 15 | 5.38 (m) | 111.4 | 44 | 3.32 (s) | 59.1 |
| 16 | 6.57 (m) | 120.7 | 45 | 3.21 (s) | 56.3 |
| 17-NH | 9.28 (d, 10.5) | — | 46 | 1.82 (br s) | 24.8 |
| 18 | — | 166.4 | 47 | 2.42 (m) | 38.4 |
| | | | | 2.26 (m) | |
| 19 | 3.39 (m) | 43.9 | 48 | — | 173.8 |
| 20 | — | 155.8 | 49 | 3.61 (s) | 51.5 |
| 21 | 2.44 (m) | 34.3 | 50 | 5.87 (br s) | 118.7 |
| 22 | 4.59 (dddd, 9.9, | 75.5 | 51 | — | 164.7 |
| | 9.9, 3.7, 3.7) | | | | |
| 23 | 1.82 (m) | 35.8 | 52 | 3.33 (s) | 57.4 |
| | 1.60 (m) | | | | |
| 24 | 3.46 (m) | 80.1 | 53 | 0.87 (d, 6.8) | 14.6 |
| 25 | 1.88 (m) | 35.2 | 54 | 3.32 (s) | 57.5 |
| 26 | 1.27 (m) | 28.9 | 55 | 0.92 (d, 7.0) | 10.5 |
| 27 | 1.58 (m) | 28.3 | 56 | 3.49 (m) | 33.7 |
| 28 | 3.21 (m) | 84.8 | 57 | — | 170.1 |
| 29 | 1.72 (m) | 40.6 | 58 | 1.08 (d, 7.0) | 13.9 |
| 30 | 4.07 (m) | 68.3 | 59 | 1.82 (d, 1.2) | 23.7 |

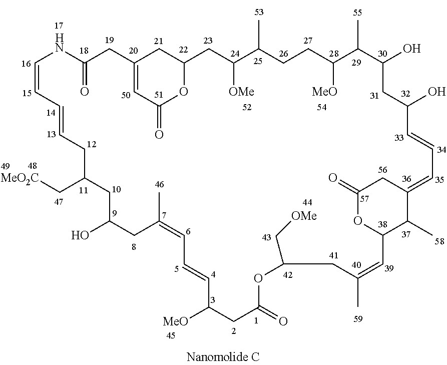

### Example 4

### Bioassays for the Detection of Antitumor Activity

The aim of this assay is to evaluate the in vitro cytostatic (ability to delay or arrest tumor cell growth) or cytotoxic (ability to kill tumor cells) activity of the samples being tested.

| Name | N^{o} ATCC | Species | Tissue | Characteristics |
|---|---|---|---|---|
| A549 | CCL-185 | human | lung | lung carcinoma (NSCLC) |
| HT29 | HTB-38 | human | colon | colorectal adenocarcinoma |
| MDA-MB- | HTB-26 | human | breast | breast adenocarcinoma |
| 231 | | | | |

### Evaluation of Cytotoxix Activity Using the SBR Colorimetric Assay

A colorimetric assay, using sulforhodamine B (SRB) reaction has been adapted to provide a quantitative measurement of cell growth and viability (following the technique described by Skehan et al. J. Natl. Cancer Inst. 1990, 82, 1107-1112).

This form of assay employs SBS-standard 96-well cell culture microplates (Faircloth et al. Methods in Cell Science, 1988, 11(4), 201-205; Mosmann et al, Journal of Immunological Methods, 1983, 65(1-2), 55-63). All the cell lines used in this study were obtained from the American Type Culture Collection (ATCC) and derive from different types of human cancer.

Cells were maintained in Dulbecco's Modified Eagle Medium (DMEM) supplemented with 10% Fetal Bovine Serum (FBS), 2 mM L-glutamine, 100 U/mL penicillin and 100 U/mL streptomycin at 37° C., 5% CO₂ and 98% humidity. For the experiments, cells were harvested from subconfluent cultures using trypsinization and resuspended in fresh medium before counting and plating.

Cells were seeded in 96 well microtiter plates, at 5×10³-7.5×10³ cells per well in aliquots of 150 μL, and allowed to attach to the plate surface for 18 hours (overnight) in drug free medium. After that, one control (untreated) plate of each cell line was fixed (as described below) and used for time zero reference value. Culture plates were then treated with test compounds (50 μL aliquots of 4× stock solutions in complete culture medium plus 4% DMSO) using ten serial dilutions (concentrations ranging from 10 to 0.00262 μg/mL) and triplicate cultures (1% final concentration of DMSO). After 72 hours treatment, the antitumor effect was measured by using the SRB methodology: Briefly, cells were washed twice with PBS, fixed for 15 min in 1% glutaraldehyde solution at room temperature, rinsed twice in PBS, and stained in 0.4% SRB solution for 30 min at room temperature. Cells were then rinsed several times with 1% acetic acid solution and air-dried at room temperature. SRB was then extracted in 10 mM trizma base solution and the absorbance measured in an automated spectrophotometric plate reader at 490 nm. Effects on cell growth and survival were estimated by applying the NCI algorithm (Boyd M R and Paull K D. Drug Dev. Res. 1995, 34, 91-104).

Using the mean±SD of triplicate cultures, a dose-response curve was automatically generated using nonlinear regression analysis. Three reference parameters were calculated (NCI algorithm) by automatic interpolation: GI₅₀=compound concentration that produces 50% cell growth inhibition, as compared to control cultures; TGI=compound concentration that produces total cell growth inhibition (cytostatic effect), as compared to control cultures, and LC₅₀=compound concentration that produces 50% net cell killing (cytotoxic effect).

Table 4 illustrates data on the biological activity of compounds of the present invention.

TABLE 4
Cytotoxicity assay-Activity Data (Molar) of Nanomolides A-C.

| | | Nanomolide A | Nanomolide B | Nanomolide C |
|---|---|---|---|---|
| MDA-MB-231 | GI₅₀ | 8.35E−09 | 9.49E−08 | 1.52E−08 |
| | TGI | 1.71E−08 | 9.78E−08 | 1.90E−08 |
| | LC₅₀ | 4.55E−08 | 1.08E−07 | 2.28E−08 |
| HT29 | GI₅₀ | 7.59E−09 | 8.80E−08 | 1.23E−08 |
| | TGI | 1.04E−08 | 9.29E−08 | 1.52E−08 |
| | LC₅₀ | 1.61E−08 | 9.78E−08 | 2.18E−08 |
| A549 | GI₅₀ | 1.04E−08 | 7.34E−08 | 1.42E−08 |
| | TGI | 1.52E−08 | 1.27E−07 | 2.47E−08 |
| | LC₅₀ | 2.37E−08 | 2.35E−07 | 4.93E−08 |

1. A compound of general formula I 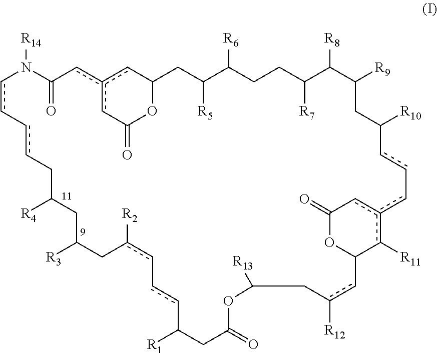
wherein
each R₁, R₃, R₅, R₇, R₉, and R₁₀ is independently selected from hydrogen, halogen, ORₐ, OCORₐ, OCOORₐ, OCONRₐR_{b}, OSO₂Rₐ, OSO₃Rₐ, and ═O, with the proviso that when a ═O group exists the hydrogen of the C atom to which the ═O is attached is absent;
each R₂, R₄, R₆, R₈, R₁₁, R₁₂, and R₁₃ is independently selected from hydrogen, CORₐ, COORₐ, CONRₐR_{b}, substituted or unsubstituted C₁-C₁₂ alkyl, substituted or unsubstituted alkenyl, and substituted or unsubstituted C₂-C₁₂ alkynyl; or R₃ and R₄ together with the corresponding C atoms to which they are attached and their adjacent C atom form a 5 or 6 membered lactone or lactam ring;
R₁₄ is independently selected from hydrogen, CORₐ, COORₐ, CONRₐR_{b}, ORₐ, OCORₐ, substituted or unsubstituted C₁-C₁₂ alkyl, substituted or unsubstituted C₂-C₁₂ alkenyl, and substituted or unsubstituted C2-C₁₂ alkynyl;
each Rₐ and R_{b} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
each [symbol] line represents a single or double bond, with the proviso that when one carbon atom bears more than one [symbol] line one of these lines can be a double bond but the others are single bonds;
or a pharmaceutically acceptable salt, tautomer, prodrug or stereoisomer thereof.

2. A compound according to claim 1, having the following formula II 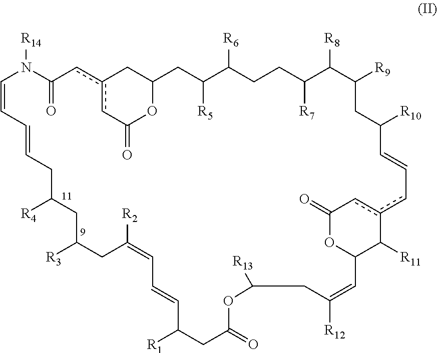
wherein R₁-R₁₄ and the [symbol] lines are as defined in claim 1, or a pharmaceutically acceptable salt, tautomer, prodrug or stereoisomer thereof.

3. A compound according to claim 1, wherein R₁, R₅, R₇, R₉, and R₁₀ are each independently selected from ORₐ, OCORₐ, and OCOORₐ, wherein Rₐ is selected from hydrogen and substituted or unsubstituted C₁-C₆ alkyl.

4. A compound according to claim 3, wherein R1, R5, and R7 are methoxy.

5. A compound according to claim 1, wherein R9 and R10 are hydroxy.

6. A compound according to claim 1, wherein R2, R6, R8, R11, and R12 are each independently selected from hydrogen and substituted or unsubstituted C1-C6 alkyl.

7. A compound according to claim 6, wherein R2, R6, R8, R11, and R12 are methyl.

8. A compound according to claim 1, wherein R13 is substituted or unsubstituted C1-C6 alkyl.

9. A compound according to claim 8, wherein R13 is a substituted C1-C6 alkyl substituted with OR′, SR′, NHR′, N(R′)2, NHCOR′, N(COR′)2, halogen, OCOR′, OCOOR′, OCONHR′, or OCON(R′)2, wherein each of the R′ groups is independently selected from hydrogen, substituted or unsubstituted C1-C6 alkyl, substituted or unsubstituted C2-C6 alkenyl, and substituted or unsubstituted C2-C6 alkynyl.

10. A compound according to claim 9, wherein R13 is methoxymethyl.

11. A compound according to claim 1, wherein R14 is selected from hydrogen and substituted or unsubstituted C1-C6 alkyl.

12. A compound according to claim 11, wherein R14 is hydrogen.

13. A compound according to claim 1, wherein R3 is selected from ORa, OCORa, and OCOORa, wherein Ra is selected from hydrogen and substituted or unsubstituted C1-C6 alkyl.

14. A compound according to claim 13, wherein R3 is hydroxy.

15. A compound according to claim 1, wherein R4 is a substituted or unsubstituted C1-C6 alkyl.

16. A compound according to claim 15, wherein R4 is a substituted C1-C6 alkyl substituted with SO2R′, COR′, COOR′, CONHR′, CON(R′)2, CON(R′)OR′, CON(R′)SO2R′, PO(OR′)2, PO(OR′)R′, PO(OR′)(N(R′)R′), or substituted or unsubstituted heterocyclic group, wherein each of the R′ groups is independently selected from hydrogen, substituted or unsubstituted C1-C6 alkyl, substituted or unsubstituted C2-C6 alkenyl, and substituted or unsubstituted C2-C6 alkynyl.

17. A compound according to claim 16, wherein R4 is methoxycarbonylmethyl.

18. A compound according to claim 1, wherein R3 and R4 together with the corresponding C atoms to which they are attached and their adjacent C atom form a 5 or 6 membered lactone ring.

19. A compound according to claim 18, wherein R3 and R4 together with the corresponding C atoms to which they are attached and their adjacent C atom form a 6 membered lactone ring of formula

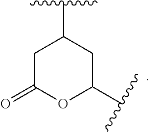

20. A compound according to claim 19, wherein the 6 membered lactone ring is of formula 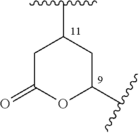
wherein the labeled C atoms correspond with their homonyms in formula I or II.

21. A compound according to claim 1, wherein each [symbol] [symbol] line is a double bond, with the proviso that when one carbon atom bears more than one [symbol] line one of these lines is a double bond and the others are single bond.

22. A compound according to claim 21, wherein at least one lactone ring has a double bond conjugated with its carbonyl group.

23. A compound according to claim 1, having the following structure: 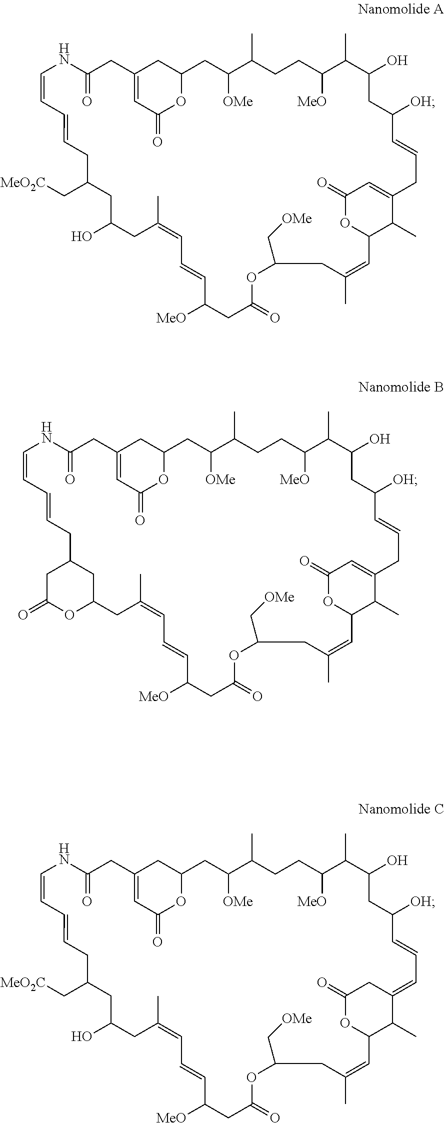
or a pharmaceutically acceptable salt, tautomer, prodrug or stereoisomer thereof.

24. A pharmaceutical composition comprising a compound according to any preceding claim, or a pharmaceutically acceptable salt, tautomer, prodrug or stereoisomer thereof, and a pharmaceutically acceptable carrier or diluent.

25. (canceled)

26. (canceled)

27. A method of treating a patient affected by cancer which comprises administering to said affected individual in need thereof a therapeutically effective amount of a compound as defined in any of claims 1 to 23.
